(19) United States
(12) Patent Application Publication
(10) Pub. No.: US 2015/0327767 A1
(43) Pub. Date: Nov. 19, 2015

(54) IMAGING METHOD FOR RESECTED MATTER AND/OR FOR A RESECTION BED AND IMAGE GENERATION DEVICE FOR CARRYING OUT SUCH AN IMAGING METHOD

(71) Applicants: CARL ZEISS AG, Oberkochen (DE); CARL ZEISS MEDITEC AG, Jena (DE)

(72) Inventors: Wibke HELLMICH, Jena (DE); Holger FUCHS, Aalen (DE); Christoph RUSSMANN, Jena (DE); Christoph NIETEN, Jena (DE); Tobias SCHMITT-MANDERBACH, Jena (DE)

(21) Appl. No.: 14/652,902

(22) PCT Filed: Dec. 18, 2013
(86) PCT No.: PCT/EP2013/077223

§ 371 Date: Jun. 17, 2015

(30) Foreign Application Priority Data
Dec. 18, 2012 (DE) 102012223651.1

(51) Int. Cl. A61B 5/00; A61N 5/06; A61M 37/00; A61N 5/10

(52) U.S. Cl. CPC A61B 5/0095; A61N 5/1039; A61B 5/742; A61N 5/062; A61M 37/0092; A61N 2005/1098

(57) ABSTRACT

Provided is an imaging method in which a surface of resected matter is imaged in first images with a photoacoustic image generation method down to a first predetermined depth such that in the first images there is a contrast between pathological tissue, in particular cancerous tissue, and non-pathological tissue.

### PRIORITY

This application claims the benefit of German Patent Application No. 102012223651.1, filed on Dec. 18, 2012, which is hereby incorporated herein by reference in its entirety.

### FIELD

The present invention relates to an imaging method for resected matter and/or for a resection bed as well as an image generation device for carrying out such an imaging method.

### BACKGROUND

Hitherto, laborious instantaneous section examinations have frequently been carried out to detect pathological tissue (in particular cancerous tissue) in resected matter. With such an instantaneous section examination, which takes at least 20 to 30 minutes and which cannot be accelerated for example by carrying out individual steps of the instantaneous section examination in parallel, since these steps are to be carried out one after the other, only first histological examinations can be carried out, however. The operation is interrupted while the resected matter is examined in an external laboratory and only continued when the results of the instantaneous section examination are available. However, final results are not delivered on the examination of resected matter but often take days. Therefore, only the results of the instantaneous section examination can be used as an information basis for continuing or stopping the operation. An additional uncertainty arises because only part of the resected matter can be examined by means of the frozen section examination within the short time. When the operation is continued there is the risk of operating over a wider area, whereas when the operation is stopped there is the risk of a reoperation—in both cases an extremely unsatisfactory solution for the patient.

Conventional fluorescent methods are limited with regard to determining pathological tissue by their small penetration depth. In many countries there are specifications on how large the area of resected matter which is free from cancerous cells must be. In Germany, for example, the interdisciplinary S3-Guideline for the Diagnosis, Therapy and Follow-up of Breast Cancer in the update of 2012, depending on the type of the tumor, specifies a minimum safety distance of 1 mm (or at least 2 mm for a ductal carcinoma in situ), in order to guarantee quality assurance of the operative procedure.

### SUMMARY

Disclosed is an imaging method with which pathological tissue can be clearly displayed down to a predetermined first depth from the surface of the resected matter. Furthermore, a corresponding image generation device is disclosed.

Disclosed is an imaging method in which (preferably all of) the surface of the resected matter is imaged in first images with a photoacoustic image generation method down to a first predetermined depth such that there is a contrast in the first images between pathological tissue, in particular cancerous tissue, and non-pathological tissue.

By using photoacoustic image generation, the surface of the resected matter can be imaged down to a sufficient first predetermined depth with the result that information is available which is better in terms of content and is quicker (compared with an instantaneous section examination), which information can be used e.g. to decide whether an operation must be continued or whether it can be stopped. It is thus advantageously achieved that the interruption of the operation (during which the surface of the resected matter is imaged in the first images) and thus the stress for the patient can be reduced.

A bowl-shaped volume area of the resected matter can be imaged photoacoustically, wherein the thickness of the bowl corresponds to the first predetermined depth.

At least one contrast agent, which increases the contrast, can be administered to the resected matter. The at least one contrast agent can be administered as first contrast agent after removal of the resected matter and/or as second contrast agent before removal of the resected matter. The same contrast agent or different contrast agents can be used as first contrast agent and as second contrast agent.

The first contrast agent (and/or the second contrast agent) can be used in particular for the visualization of an increased vascularization. In this case, a first contrast agent is chosen with which e.g. a specific staining of the vessels (for example vessel walls, endothelial cells, etc.) is possible.

The second contrast agent (and/or the first contrast agent) can, in particular, be a contrast agent which accumulates specifically in cancerous tissue. In this way, a very exact imaging of cancerous tissue can be carried out within the first predetermined depth.

The second contrast agent, which is administered before the removal of the resected matter, can be administered for example intravenously to the person or animal from whom the resected matter is taken.

The resected matter is, in particular, tissue from a human or animal body, e.g. a tumor (e.g. a lung tumor, a thyroid tumor, an ovarian tumor, a skin tumor, a brain tumor, a retinal tumor, a tumor in the gastrointestinal tract, a lymph node tumor, a prostate tumor and/or a cervical tumor) and/or tissue from a breast of a female body, such as e.g. a breast tumor.

As contrast agent, e.g. probes (e.g. antibody-dye complexes or antibody-fluorescent dye complexes) which bind specifically to at least one antigen can be used. The antigen can be a tumor-specific antigen. Examples of such antigens are ER, PR, HER2, CA15-3, CA27.29, GCDFP-15, NSE, M2-PK and HER2. Thereby e.g. a tumor-specific marking is possible. These probes are particularly suitable as second contrast agent. Those which are on the surface of the tumor cell can in particular be used as antigen or marker.

In principle, suitable contrast agents can be used which are known from the field of immunomarking. Dyes or fluorescent dyes which bind non-specifically to e.g. the tumor cells can also be used as contrast agent. Examples are SF64, ICG and TSG. These are particularly suitable as second contrast agent.

The photoacoustic image generation method can include exposing the resected matter to a first electromagnetic radiation with a first wavelength in order to generate first pressure waves, wherein the generated first pressure waves are detected in order to generate the first images based on the detected first pressure waves. In particular, the first wavelength can be chosen depending on the at least one contrast agent. The wavelength is, in particular, chosen such that the contrast agent has a very high absorptance for this wavelength. Thus, the contrast agent can have a first wavelength range with an absorptance lying above a predetermined threshold value and the first wavelength can be chosen such that it lies in the first wavelength range. The first wavelength can be in the range of from 400 nm to 3 μm, preferably 400 nm to 1300 nm, 400 nm to 1000 nm, 400 nm to 700 nm, 700 nm to 3 μm, 700 nmto 1300 nm or 700 nm—1000 nm, for example.

A three-dimensional recording can be generated from the first images. With such a three-dimensional recording it is possible to visualize clearly whether, and if so, where there is pathological tissue in the resected matter within the first predetermined depth.

The first predetermined depth can be, for example, in the range of from (preferably greater than) 0 mm (=lower limit) to less than or equal to 10 mm (=upper limit). The lower limit can be e.g. 0.5 mm, 1 mm, 2 mm, 3 mm, 4 mm, 5 mm, 6 mm, 7 mm or 8 mm and the upper limit can be e.g. 10, 9, 8, 7, 6, or 5 mm, wherein in each of the possible ranges the lower limit is smaller than the upper limit.

In addition to, or as an alternative to, the imaging of the resected matter, a resection bed from which the resected matter was removed can be imaged down to a second predetermined depth with a photoacoustic image generation method in second images such that there is a contrast in the second images between pathological tissue, in particular cancerous tissue, and non-pathological tissue.

The second predetermined depth can be, for example, in the range of from (preferably greater than) 0 mm (=lower limit) to less than or equal to 10 mm (=upper limit). The lower limit can be e.g. 0.5 mm, 1 mm, 2 mm, 3 mm, 4 mm, 5 mm, 6 mm, 7 mm or 8 mm and the upper limit can be e.g. 10, 9, 8, 7, 6, or 5 mm, wherein in each of the possible ranges the lower limit is smaller than the upper limit.

For the resection bed, it is thus also possible to determine, within a safety margin (the second predetermined depth) and thus in a bowl-shaped volume area, the thickness of which corresponds to the second predetermined depth, whether there is still pathological tissue present or not. This can also then be used in the decision (for example by a surgeon) of whether the operation must be continued or whether it can be concluded.

A medicament can be administered to the resection bed, the effect of which is only activated by exposure to a predetermined type of radiation, wherein the pathological tissue (preferably locally, in a selective manner based on the second images) is exposed to the predetermined type of radiation in order to (preferably locally, in a targeted manner) activate the effect of the medicament (e.g. by releasing the active ingredient of the medicament).

In order to achieve the desired local effect of the medicament, in general either the medicament can be exposed locally and/or the predetermined type of radiation can be used to activate the medicament locally. Thus it is e.g. also possible only to expose the medicament locally and to direct the predetermined type of radiation onto a wider area.

The predetermined type of radiation can be, in particular, ultrasound, electromagnetic radiation (in particular from the visible wavelength range into the infrared range, e.g. with a wavelength from the range of from 400 nm to 3 μm, preferably 400 nm to 1300 nm, 400 nm to 1000 nm, 400 nm to 700 nm, 700 nm to 3 μm, 700 nm to 1300 nm or 700 nm—1000 nm) or ionizing radiation, for example.

The medicament can be the first and/or second contrast agent. In this case, the exposure to the medicament can be carried out in a separate step or the exposure to the contrast agent which has already been carried out realizes the step of administering the medicament at the same time.

The pathological tissue in the resection bed can be selectively exposed to ionizing radiation based on the second images using a probe. In this case, the step of administering the medicament can also be omitted.

Also disclosed is an image generation device for carrying out the imaging method disclosed herein. The device includes an optics module for exposing the surface of the resected matter to electromagnetic radiation, an acoustics module for detecting the generated pressure waves, a holding device for relative movement between the optics module and the resected matter, and an image generation module, which generates the first images based on the detected pressure waves.

The image generation device can, in particular, be developed in such a way that it can carry out the steps described in connection with the imaging method disclosed herein (including further development thereof).

The imaging method can include the steps which are described in connection with the image generation device according to the invention (including further developments thereof).

It is understood that the features mentioned above and those yet to be explained below can be used not only in the stated combinations but also in other combinations or alone, without departing from the scope of the present invention.

The above summary is not intended to limit the scope of the invention, or describe each embodiment, aspect, implementation, feature or advantage of the invention. The detailed technology and preferred embodiments for the subject invention are described in the following paragraphs accompanying the appended drawings for people skilled in this field to well appreciate the features of the claimed invention.

### BRIEF DESCRIPTION OF THE DRAWINGS

FIG. 1 is an image generation device according to certain embodiments.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION

In the following descriptions, the present invention will be explained with reference to various exemplary embodiments. Nevertheless, these embodiments are not intended to limit the present invention to any specific example, environment, application, or particular implementation described herein. Therefore, descriptions of these example embodiments are only provided for purpose of illustration rather than to limit the present invention.

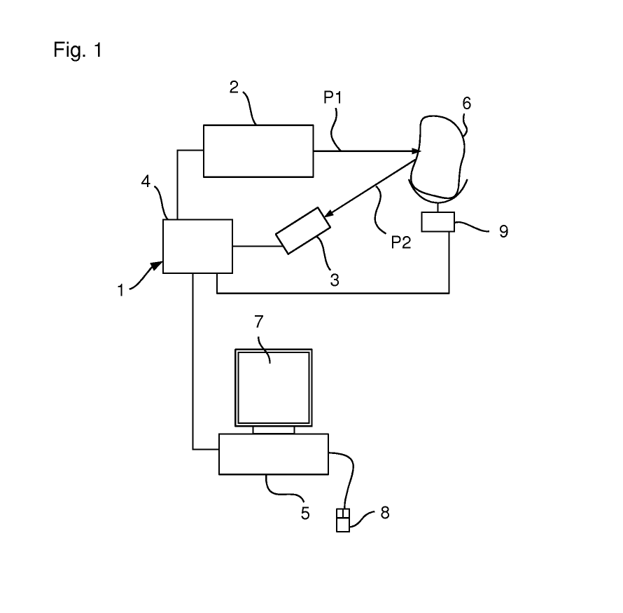

In FIG. 1 an embodiment of an image generation device 1 according to the invention is shown schematically, which comprises an optics module 2, an acoustics module 3, a holding device 9, a control module 4 to control the optics and acoustics modules 2,3 and the holding device 9, as well as optionally an input module 5.

The image generation device 1 here is configured such that, with it, images of the surface (in particular the entire surface) of resected matter 6 (e.g. a tumor which was removed in a manner which conserved the breast) are generated down to a predetermined depth such that in the images there is a contrast between pathological tissue, in particular cancerous tissue, and non-pathological tissue. For this, the resected matter 6 can be exposed by means of the optics module 2 to e.g. pulsed electromagnetic radiation in the near infrared range (700-3 μm), as is indicated by the arrow P1, and the pressure waves generated in this way can be detected by means of the acoustics module 3, as is indicated by the arrow P2. In this way, the photoacoustic effect is used for image generation, wherein the excitatory electromagnetic radiation is focused, for example, on the areas of the resected matter to be imaged (e.g. in a diffraction limited manner) and is moved in this location in order to generate an image point by point. Additionally or alternatively, a relative movement (e.g. rotation) between the resected matter 6 and the optics module 2 or the electromagnetic radiation emitted by the latter can be generated by means of the schematically represented holding device 9, in order thus to be able to generate images of the entire surface of the resected matter 6. To generate this relative movement, the optics module 2 and/or the resected matter 6 can be moved by means of the holding device. Furthermore, if necessary, the acoustics module 3 (or at least the corresponding detector of the acoustics module 3) can also be moved. During the image generation, the resected matter 6 can lie e.g. in a waterbed (not shown, which is provided e.g. by the holding device 9), in order to achieve a good acoustic coupling to the acoustic detection.

The image generation device 1 can also be configured such that a tomographic image generation can be performed. The resected matter 6 is exposed by means of the optics module 2 to e.g. pulsed electromagnetic radiation in the near infrared range and the induced pressure waves are recorded by a detector rotating about the resected matter 6 and/or a detector array with subsequent reconstruction of the data to form a tomographic image. The rotating detector or detector array is part of the acoustics module 3. In this case, the resected matter 6 no longer needs to be rotated but is pushed along a first axis through the illuminated or detector area (as with a conventional computer tomograph). Alternatively or additionally, the illumination (optics module 2) and the detector can be displaced. If the entire surface of the resected matter is intended to be covered, the resected matter can still be rotated about the first axis.

The image generation device 1 can furthermore be configured such that both variants described (illumination with the moved focused spot and the tomographic image generation) are possible. Images of both recording variants can then be obtained and assessed. Optionally, a combination of the image data is possible.

The input module 5 can e.g. serve to select the desired measurement program and/or to display the generated image and can, for example, be realized as a conventional computer and have a monitor screen 7 and an input unit 8, which is represented here schematically as a computer mouse. The input unit 8 can additionally or alternatively comprise a keyboard or other operating panel. Furthermore, it is possible, additionally or alternatively, for the monitor screen 7 itself to be realized as input unit 8. For example, the monitor screen 7 can be touch-sensitive.

With the image generation device 1, images of the entire surface of the resected matter 6 can be generated down to a predetermined depth (which e.g. can be approx. 10 mm). By means of these images, it is then possible to determine whether cancerous tissue is still present or not up to the surface of the resected matter or within the predetermined depth from the surface of the resected matter 6. If no more cancerous tissue can be detected or imaged within the predetermined depth, which can also be called safety margin, it can be assumed that sufficient tissue was removed to remove the tumor. If cancerous tissue is still imaged, it can be necessary to remove additional tissue from the breast in order to achieve the removal of all tumor cells.

In order to achieve good contrast in the photoacoustic image generation, the resected matter 6 can be exposed to a first contrast agent after the removal. The exposure thus takes place ex vivo and can, for example, serve to stain the increased vascularization present as a result of the pathological tissue.

In addition to the photoacoustic imaging of the entire surface of the resected matter which can also be used to generate a 3D image, the resection bed (i.e. the area which remains after removal of the resected matter, here e.g. in the breast) can further be imaged photoacoustically down to a predetermined second depth in order to also generate images here within the second depth, which represents a second safety margin, which images can be used to establish whether pathological tissue is still present. The resection bed can be examined partially or as a whole.

The resection bed can be exposed to the first contrast agent in the same way as the resected matter.

It is also possible to examine the resection bed on the adjacent lymph nodes by means of optical acoustic imaging. The functionally adjacent lymph nodes can be examined in vivo or ex vivo. In particular, they are often also removed during an operation because they often contain tumor cells. The lymph vessels and nodes can, like blood vessels, be stained with a contrast agent.

As an alternative or in addition to the administration of the first contrast agent, it is possible, before the removal of the resected matter 6, to administer a second contrast agent (for example by means of an intravenous injection) and for the removal of the resected matter to take place only after a predetermined time T has elapsed. The time T is chosen such that the introduced second contrast agent has accumulated specifically in the pathological tissue.

The second contrast agent can thus preferably be chosen such that a specific accumulation takes place in pathological tissue (in particular in cancerous tissue). The first contrast agent is, in particular, chosen such that it is thereby possible to detect an increased vascularization since a higher density of blood vessels correlates with cancerous tissue. The specific excitation wavelengths (the wavelengths at which the contrast agents have a particularly high absorption) can be different for the first and second contrast agent. When both contrast agents have been administered, photoacoustic recordings are therefore preferably carried out with both excitation wavelengths.

As first and/or second contrast agent, e.g. indocyanine green (referred to below as ICG), can be used. In particular, the contrast agent can be administered as packaged contrast agent, such as e.g. ICG which is packaged in a micellar structure. The production of such a micellar ICG is described e.g. in Kirchherr et al.: Molecular Pharmaceutics 2009, volume 6, No. 2, pp. 480-491, wherein the content of the article is incorporated here in the present description. Besides ICG, any other absorber (e.g. fluorescein) which e.g. preferably has a high absorptance or absorption cross section in the near infrared range or in the infrared range can, of course, also be used. For the packaging or formation of the micellar structure e.g. Sulotol, phospholipid-polyethylene glycol or polyglycerol sulphate can be used. However, the absorber can also be packaged in a viral vector (target-specific molecule) or in a nanoparticle/nanotube with molecule-, cell- and/or tissue-specific properties.

In the variant of administering the second contrast agent after removal of the resected matter 6, the resection bed can be photoacoustically imaged in order to establish whether or not there is still pathological tissue also within the predetermined second depth within the resection bed.

If, e.g. pathological tissue is still present, a medicament (which is advantageously absorbed specifically by the pathological tissue) the active ingredient of which is preferably activated (e.g. released) only after exposure to a predetermined type of radiation can be introduced into the resection bed.

Thus, the activation of the active ingredient can be triggered by ultrasound. In this case, e.g. the acoustics module 3 can have an ultrasound source (transducer) which can also serve at the same time as detector for the photoacoustic measurement. By means of the ultrasound source, the resection bed can then be locally (in the areas with detected pathological tissue) exposed to ultrasound in order to release the active ingredient of the medicament there. Even if the medicament is not absorbed specifically by the pathological tissue, a local specific release of the active ingredient can thus be achieved.

It is also possible to activate the active ingredient by the action of light (of a predetermined wavelength). It is preferably a wavelength which can be generated by means of the optics module 2. Thus, the active ingredient of the medicament can be activated (e.g. released) site-specifically through exposure to the electromagnetic radiation or the light of the optics module 2. If another light source with another wavelength is necessary, this can e.g. be part of the image generation device 1.

As medicament e.g. the first and/or second contrast agent can be used, wherein at least in this case the first and/or second contrast agent preferably has a phototoxic effect. The introduction of the medicament can, as already described, be carried out in a separate step.

However, it is also possible for the first and/or second contrast agent, which was already introduced for the photoacoustic imaging, also to be used as medicament. In this case, a separate step of introducing the medicament is not necessary.

The same light source as is used to generate the pressure waves for the photoacoustic detection can also be used for the activation of the medicament. If necessary, only the light intensity, distribution and/or wavelength is to be adjusted.

Activation of the active ingredient (e.g. release of the active ingredient) is also possible as a reaction to the exposure to ionizing radiation. In this case, a corresponding actinotherapeutic probe is used to emit the ionizing radiation. The probe which emits the ionizing radiation can also be used directly (i.e. without administering a medicament) and the detected pathological tissue in the resection bed can be irradiated with it selectively. The probe can be part of the image generation device 1.

The image generation device 1 disclosed herein and the methods disclosed herein possess the advantage that a volume, and no longer as previously only a frozen section, of the resected matter can be examined in a shorter time. The preparation work in the method according to the invention is also significantly lower than in the case of a frozen section examination. In this way, the duration of the interruption of the operation which was previously necessary to wait for the result of a frozen section can be significantly reduced and thus the stress for the patient can also be reduced. Better conclusions can also be drawn since the entire surface of the resected matter can be examined.

If both contrast agents are used, there is double proof since, on the one hand, the blood vessel density and, on the other hand, the pathological tissue itself can be imaged with high contrast.

The generated images can, for example, be preprocessed using suitable algorithms, e.g. noise can be removed from them and/or they can be compressed.

Furthermore, it is possible to lay images with different modalities (e.g. images with the first and the second contrast agent and also images from other imaging methods) over each other in order to thus make better diagnoses possible. To achieve such a superimposition, suitable registration algorithms are used.

The generated images can be assessed semi-automatically or automatically. The safety margin can, e.g. be visualized. The visualization can, in particular, take place via a false color representation. Thus, for example, red can be used to show that there is not a sufficient safety margin (which can e.g. be 5 mm) or from where pathological tissue is present, yellow characterizes the limit range and green indicates that there is no pathological tissue in the safety margin or where there is no pathological tissue.

The acoustics module 3 can, for example, include a single ultrasound detector, several ultrasound detectors or even an ultrasound detector array. With several detectors it is more easily possible to record images more quickly and/or to produce a tomograph.

The recorded data or images can be stored for documentation purposes and/or for comparison with other histological, biochemical and/or imaging methods. In particular, e.g. a differential diagnosis, validation of initial findings or therapy control can be carried out based on these data/images. The method according to the invention can contain the step of removing the resected matter.

The electromagnetic radiation of the optics module 2 can, in particular, be laser radiation. The electromagnetic radiation or the laser radiation can be pulsed and/or intensity modulated.

As has already been described, the resected matter 6 can be laid in a waterbed in order to achieve a good acoustic detection of the generated sound waves. Of course, detection is also possible without such a waterbed. In particular, e.g. a contactless detection is also possible by detecting the change of position of the corresponding surface section of the resected matter.

The image generation device 1 can be extremely compact. In particular, parts of the image generation device 1 can be configured as portable parts or as hand-held probes or probe heads. This is particularly advantageous in the examination of the resection bed and in the targeted exposure to release the active ingredient of the medicament.

The generated images can be adjusted individually. A representation of the data without comments, and thus a clean image representation, is possible. Furthermore, additional information and false colors can be used in order to provide further information.

The image data processing and representation can, for example, be realized by means of the input module 5.

Furthermore, it is possible to generate three-dimensional image data sets and tomograms from the recorded images. Furthermore, a comparison of the generated images or image data sets or tomograms can be carried out with image data sets or other information from an imaging carried out before the operation. This can, in particular, be a computer tomogram, a magnetic resonance tomogram, an ultrasound image, etc.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

1-15. (canceled)

16. An imaging method, comprising:
imaging a surface of a resected matter in a first set of images via photoacoustic image generation down to a first predetermined depth such that in the first set of images there is a contrast between pathological tissue and non-pathological tissue.

17. The imaging method of claim 16, wherein a bowl-shaped volume area of the resected matter is imaged via photoacoustic image generation, and wherein the thickness of the bowl-shaped volume area corresponds to the first predetermined depth.

18. The imaging method of claim 17, further comprising administering at least one contrast agent to the resected matter to increase the contrast.

19. The imaging method of claim 18, wherein the at least one contrast agent is an encapsulated contrast agent.

20. The imaging method of claim 18, wherein at least one of the at least one contrast agent is administered as first contrast agent after removal of the resected matter.

21. The imaging method of claim 18, wherein at least one of the at least one contrast agent is administered as second contrast agent before removal of the resected matter.

22. The imaging method of claim 18, wherein the resected matter is exposed to a first electromagnetic radiation with a first wavelength in order to generate first pressure waves, the method further comprising selecting the at least one contrast agent to define the first wavelength.

23. The imaging method of claim 16, further comprising administering at least one contrast agent to the resected matter to increase the contrast.

24. The imaging method of claim 23, wherein the at least one contrast agent is an encapsulated contrast agent.

25. The imaging method of claim 23, wherein at least one of the at least one contrast agent is administered as first contrast agent after removal of the resected matter.

26. The imaging method of claim 23, wherein at least one of the at least one contrast agent is administered as second contrast agent before removal of the resected matter.

27. The imaging method of claim 23, wherein the resected matter is exposed to a first electromagnetic radiation with a first wavelength in order to generate first pressure waves, the method further comprising selecting the at least one contrast agent to define the first wavelength.

28. The imaging method of claim 16, further comprising generating a three-dimensional recording from the first images.

29. The imaging method of claim 16, further comprising:
removing at least some of the resected matter to form a resection bed; and
imaging a surface of the resection bed in a second set of images by imaging down to a second predetermined depth via photoacoustic image generation such that in the second set of images there is a contrast between pathological tissue and non-pathological tissue.

30. The imaging method of claim 29, wherein a bowl-shaped volume area of the resection bed is imaged via photoacoustic image generation, and wherein the thickness of the bowl-shaped volume area of the resection bed corresponds to the second predetermined depth.

31. The imaging method of claim 30, further comprising:
administering a medicament to the resection bed; and
exposing the pathological tissue to the predetermined type of radiation in order to activate an effect of the medicament.

32. The imaging method of claim 31, further comprising at least one of:
selectively administering the medicament locally to the pathological tissue based on the second images; and
selectively exposing the pathological tissue locally to the predetermined type of radiation based on the second images.

33. The imaging method of claim 31, wherein the predetermined type of radiation is a member of the group consisting of ultrasound, electromagnetic radiation or ionizing radiation.

34. The imaging method of claim 29, further comprising:
administering a medicament to the resection bed; and
exposing the pathological tissue to the predetermined type of radiation in order to activate an effect of the medicament.

35. The imaging method of claim 34, further comprising at least one of:
selectively administering the medicament locally to the pathological tissue based on the second images; and
selectively exposing the pathological tissue locally to the predetermined type of radiation based on the second images.

36. The imaging method of claim 34, wherein the predetermined type of radiation is a member of the group consisting of ultrasound, electromagnetic radiation or ionizing radiation.

37. The imaging method of claim 29, further comprising selectively exposing the pathological tissue in the resection bed to ionizing radiation with a probe based on the second images.

38. Image generation system for carrying out an imaging method, the imaging method comprising imaging a surface of a resected matter in a first set of images via photoacoustic image generation down to a first predetermined depth such that in the first set of images there is a contrast between pathological tissue and non-pathological tissue, the system comprising:
an optics module to expose the surface of the resected matter to electromagnetic radiation;
an acoustics module to detect the generated pressure waves;
a holder configured to provide relative movement between the optics module and the resected matter; and
an image generator configured to generate the first images based on the detected pressure waves.
